(19) United States
(12) Patent Application Publication
(10) Pub. No.: US 2024/0285704 A1
(43) Pub. Date: Aug. 29, 2024

(54) COMPOSITIONS COMPRISING ORGANIC MINERAL CHELATES, NIACINAMIDE, AND HEMP OIL AND USES THEREOF FOR NEUROPROTECTION, CARDIOPROTECTION, DETOXIFICATION, IMMUNE SUPPORT, AND ANTI-AGING

(71) Applicant: FP Nutraceuticals, LLC, Santa Fe Springs, CA (US)

(72) Inventor: Timothy M. Marshall, Tucson, AZ (US)

(21) Appl. No.: 18/406,671

(22) Filed: Jan. 8, 2024

Related U.S. Application Data
(63) Continuation of application No. 17/157,701, filed on Jan. 25, 2021, now abandoned.
(60) Provisional application No. 62/965,440, filed on Jan. 24, 2020.
(60) Provisional application No. 62/971,023, filed on Feb. 6, 2020.

(51) Int. Cl. A61K 36/185; A23L 33/105; A23L 33/115; A23L 33/125; A23L 33/155; A23L 33/16; A23L 33/165; A23L 33/175; A61K 9/00; A61K 31/015; A61K 31/05; A61K 31/07; A61K 31/122; A61K 31/145; A61K 31/16; A61K 31/192; A61K 31/194; A61K 31/198; A61K 31/232; A61K 31/28; A61K 31/30; A61K 31/315; A61K 31/355; A61K 31/375; A61K 31/385; A61K 31/4188; A61K 31/455; A61K 31/51; A61K 31/525; A61K 31/592; A61K 31/593; A61K 31/675; A61K 31/69; A61K 31/714; A61K 33/06; A61K 33/18; A61K 36/03; A61K 36/42; A61K 36/45

(52) U.S. Cl. CPC A61K 36/185; A23L 33/105; A23L 33/115; A23L 33/125; A23L 33/155; A23L 33/16; A23L 33/165; A23L 33/175; A61K 9/0056; A61K 31/015; A61K 31/05; A61K 31/07; A61K 31/122; A61K 31/145; A61K 31/16; A61K 31/192; A61K 31/194; A61K 31/198; A61K 31/232; A61K 31/28; A61K 31/30; A61K 31/315; A61K 31/355; A61K 31/375; A61K 31/385; A61K 31/4188; A61K 31/455; A61K 31/51; A61K 31/525; A61K 31/592; A61K 31/593; A61K 31/675; A61K 31/69; A61K 31/714; A61K 33/06; A61K 33/18; A61K 36/03; A61K 36/42; A61K 36/45; A23V 2002/00

(57) ABSTRACT

The compositions of this disclosure provide broad-spectrum nutrient supplementation to support the health of multiple organ and tissue systems in which mitochondrial function plays a fundamental role. In particular, the formulations promote neuroprotective, cardioprotective, immunosupportive, and anti-aging benefits. Also provided herein are novel, neuroprotective preparations containing a combination of broad- or full-spectrum hemp oil extract combined with niacinamide, a non-flushing form of vitamin B3, and methods of use are described allowing for safe, effective, and convenient use of a neuroprotective, antioxidant preparation for general and clinical use.

### TECHNICAL FIELD

This disclosure relates to novel, multi-nutrient preparations containing organic mineral chelates (e.g., glycinates, malates, taurinates) for re-establishing healthy mitochondrial function, and for optimizing neurological, cardiovascular, and immune system function for broad-spectrum anti-aging, antioxidant benefits, and multisystem protective effects.

This disclosure also relates to novel hemp oil, niacinamide preparations utilizing this unique synergy for wide-ranging neuroprotective, neurotrophic, anti-inflammatory, and anti-aging benefits. The synergistic effects of broad-(no THC) or full-spectrum (<0.3% THC) hemp oil coupled with niacinamide is utilized to reduce oxidative stress, improve cognitive function, reduce pain and inflammation, and promote general health and well-being.

This disclosure also relates to novel, neuroprotective preparations containing a combination of broad- or full-spectrum hemp oil extract combined with niacinamide, a non-flushing form of vitamin B3. In particular, the invention relates to preparations of various extracts from the hemp plant containing a broad or full spectrum of cannabinoids in combination with niacinamide in a neuroprotective blend for highly efficient oral absorption (e.g., gingival, buccal), vaporization (inhalation), and topical delivery.

Given the similar boiling point (vaporization) temperatures for hemp oil and niacinamide, and their lack of harmful degradation byproducts, makes this combination especially well-suited for electronic vaporization delivery. With their close and relatively low vaporization temperatures, niacinamide (bp 150-160° C.) and hemp-derived cannabinoids (e.g., cannabidiol, bp 160-180° C.) are ideally suited for administration via portable electronic vaporization devices, which routinely operate between 100-250° C. Note: a temperature of approximately 200° C. is sufficient for the decarboxylation, activation, and vaporization of a broad spectrum of cannabinoids.

### BACKGROUND

Vitamins and minerals serve as important cofactors and catalysts for thousands of enzymatic reactions and chemical processes in the human body. They are indispensable to life, and in health maintenance and disease prevention, but are often deficient due to a variety of factors. Nutrient-depleted soils, unbalanced diets, increased requirements due to stress, injury, excess refined sugar, caffeine, or alcohol consumption, individual idiosyncrasies, poor appetite, dieting to promote weight loss, and certain pharmaceutical medications can inhibit nutrient absorption and/or increase excretion leading to nutrient deficiencies.

Many circumstances contribute to the level of one or more vitamins or minerals may be insufficient, leading to compromised biological processes and signs of deficiency. Symptoms indicative of suboptimal nutrient intake (e.g., copper and/or zinc) include fatigue, poor stamina, cognitive impairment, cardiovascular dysfunction, skin problems, weakened immune system, and susceptibility to infections. Other common signs of vitamin and/or mineral deficiency (e.g., magnesium, zinc, B-vitamins) include digestive disturbances, hair loss, impaired wound healing, and sleep disturbances. Malabsorption of vitamins and minerals is also seen in a variety of conditions. Examples are irritable bowel syndrome, prolonged diarrhea, pernicious anemia, disorders of the liver and digestive system, prolonged diarrhea, and hyperthyroidism. In addition, since a number of vitamins (e.g., biotin, vitamin K2) are provided by the gastrointestinal microbiome, use of antibiotics that destroy beneficial bacterial flora may inevitably lead to decreased intestinal vitamin production.

The wide-ranging effects of nutrient deficiencies on multiple health outcomes has been extensively studied and described in hundreds of international studies. It has been well-documented that several key, neuroprotective, cardioprotective, immune strengthening nutrients are commonly deficient due to mineral-depleted soils and the Standard American Diet (SAD), and are further depleted by chronic stress, physical work, exercise, pregnancy, excess refined sugar, alcohol, caffeine, nicotine, glyphosate (e.g., divalent (2+) ions: magnesium, zinc, iron, copper, manganese, selenium), and various medications that inhibit vitamin absorption (e.g., proton pump inhibitors, antacids, metformin; vitamin B12), and promote the excretion of protective elements (e.g., spironolactone, copper). For example, fructose and sucrose inhibit copper absorption, and the broad-spectrum divalent metal chelating herbicide, glyphosate, used in increasingly greater amounts over the past two decades, binds to and prevents copper absorption and utilization, promoting the deficiency of copper and other essential divalent minerals (e.g., zinc, manganese).

As a population, we are starved for nutrients, especially essential minerals. United States and UK Government statistics show a decline in trace minerals up to 76% in fruit and vegetables over the period from 1940 to 1991. In a 2007 review article in the journal Nutrition and Health, titled “The Mineral Depletion Of Foods Available To Us As A Nation (1940-2002)”, the author states, “The character, growing method, preparation, source and ultimate presentation of basic staples have changed significantly to the extent that trace elements and micronutrient contents have been severely depleted. Ongoing research clearly demonstrates a significant relationship between deficiencies in micronutrients and physical and mental ill health.

Through the years, minerals are taken up by crops and inadequately returned to the soil. This has led to massive declines in a broad spectrum of essential minerals such as zinc, copper, iron, magnesium, lithium, and many others. Coupled with increasing use of toxic agricultural chemicals such as glyphosate (Roundup)—mineral depletion has gotten worse. As a powerful chelating agent, glyphosate binds to divalent metals, promoting deficiencies in a wide range of essential minerals by reducing mineral bioavailability to plants and animals. A 2013 article in Reuters titled, Heavy use of herbicide Roundup linked to health dangers—U.S. study—states, “In 2007, as much as 185 million pounds of glyphosate was used by U.S. farmers, double the amount used six years ago, according to Environmental Protection Agency (EPA) data.” Along with glyphosate's mineral-depleting effects, we know that fluoride—a known neurotoxin and mitochondrial poison can bind to the essential trace element, lithium forming an insoluble precipitate—decreasing its bioavailability to plants, animals, and humans.

The increased production of reactive oxygen species (ROS), and unabated oxidative stress due to suboptimal, deficient intake of specific antioxidant nutrients (e.g., magnesium, zinc, copper, selenium, vitamins C, E, niacin) and plant-derived antioxidants (e.g., polyphenolics, cannabinoids) is a pathophysiological pathway common to a host of chronic disease states, and primary and secondary neurological conditions. These include but are not limited to: depression, sensitivity to stress, chronic fatigue, cognitive dysfunction, mitochondrial dysfunction, sleep disturbances, high blood pressure, inflammatory skin conditions (e.g., acne, eczema, dermatitis, psoriasis), neurodegenerative diseases, chronic pain, and chronic inflammation. These disease states share the following common physiological characteristics: oxidative stress from excessive unopposed free-radical generation, elevated inflammatory biomarkers (e.g., CRP), excessive stimulation of post-synaptic neurons (N-methyl-D-aspartate receptor mediated; NMDA-R), primarily from the excitatory amino acid neurotransmitters, aspartate and glutamate, and chronic inflammation.

Common antioxidant, nutrient deficiencies play a strong role in their etiology. Magnesium, zinc, B-complex (e.g., vitamin B3), and endocannabinoid deficiencies contribute to increased production of ROS, oxidative stress, inflammation, and NMDA receptor hyperactivity. This can lead to the overstimulation of the post-synaptic neuron leading to a long-standing partially depolarized state, which opens cell membrane calcium channels, allowing an influx of calcium ions and increasing intracellular calcium concentrations.

Calcium is a cofactor for a plethora of intracellular enzymes, including proteases, DNA and RNA endonucleases, and phospholipases. Intracellular hypercalcemia resulting from overstimulation may lead to widespread activation of these enzymes causing destruction of cellular proteins, nucleic acids, and membrane structures resulting in eventual neuronal death. An optimal, therapeutic neuroprotective intervention to prevent and/or treat suboptimal antioxidant nutrient intake, and unabated oxidative stress would ideally create a microenvironment wherein oxidative stress is reduced.

Mitochondrial dysfunction and unabated oxidative stress due to nutritional deficiencies and toxin exposure (e.g., heavy metals) and accumulation are now regarded as primary contributing factors in chronic disease. The health of an individual is a direct function of their cellular redox status (e.g., antioxidant nutrient levels, tissue concentration of exogenous and endogenous antioxidants,), the health of their mitochondria, and the number of healthy mitochondria they have in their organs and tissues. Mitochondria are concentrated in the major organs and tissues and require a broad-spectrum of nutrients such as vitamins A, B-complex vitamins, C, D, E, magnesium, zinc, copper, iron, manganese, selenium, coenzyme Q10, and plant-derived antioxidants (e.g., terpenes, phenolics, cannabinoids) for optimal function and protection again free-radical generated cellular injury, otherwise known as oxidative stress. Due to the fact that mitochondria function as the oxidation furnaces of the cell (e.g., oxidative phosphorylation), whereby electrons are ultimately extracted from protein, carbohydrates, fats, and ketone bodies for energy—there is a tremendous need for a plentiful supply of antioxidants to counteract the huge number of free-radicals that are generated from oxidative metabolism.

In addition to oxidative metabolism, free radicals are produced by heavy metals such as arsenic, cadmium, lead, and mercury, a variety of organic chemicals (e.g., organochlorides, polychlorinated biphenyls), and some widely used OTC (e.g., acetaminophen; promotes glutathione depletion) and prescription medications (e.g., antibiotics). Antibiotic overuse is a primary source for mitochondrial dysfunction and injury.

Clinical use of several classes of bacteriocidal antibiotics is associated with moderate to severe side effects due to the promotion of oxidative, free-radical mediated injury and mitochondrial toxicity. Agents that counteract oxidative stress such as specific antioxidant nutrients (e.g., copper, selenium), nutraceuticals (e.g., coenzyme Q10) and plant-based antioxidants (e.g., terpenes, polyphenolics, cannabinoids) are needed to reduce mitochondrial damage and toxicity.

Unabated oxidative stress due to suboptimal intake of specific mitochondrial nutrients (e.g., vitamins A, B-complex vitamins, C, D, E, magnesium, zinc, copper, iron, manganese, selenium, glycine, taurine), plant-derived antioxidants (e.g., terpenes, phenolics) and chemical-induced toxicity and the associated increase in free-radical generation and inflammation—is a pathophysiological pathway common to a host of chronic diseases (e.g., cognitive impairment, cardiovascular disease, immune dysfunction) and premature and accelerated aging.

Because nutrient deficiencies are highly prevalent in the United States and elsewhere, appropriate supplementation and/or an improved diet could reduce much of the consequent risk of chronic disease and premature aging. The consumption and associated levels of chromium, copper, magnesium, zinc, B-complex, vitamins A, D, E, K, glycine and taurine are inadequate in a large percentage of the American population, and these deficiencies are a major contributor to a wide variety of chronic diseases and unhealthy (accelerated) aging.

There are more than 80,000 new chemicals prevalent throughout our food, air, water, cosmetics, cleaning products, hygiene products, and medicines today that were unknown to our ancestors over 100 years ago. Many of these synthetic, man-made chemicals such as glyphosate, polychlorinated biphenyls (PCBs), and pharmaceuticals interfere with nutrient absorption, retention, or metabolism, and promote the depletion of vital protective nutrients. Examples include: proton pump inhibitors, which have been shown to deplete the immunosupportive, cardioprotective, neuroprotective, anti-aging nutrients: magnesium and cobalamin; H2-antagonists, which have been shown to deplete the immunosupportive, cardioprotective, neuroprotective, anti-aging nutrients: iron, cobalamin, folic acid, vitamin D; and statins, which have been shown to deplete the immunosupportive, cardioprotective, neuroprotective, anti-aging nutrients: selenium, vitamin K2, and coenzyme Q10.

These nutrients serve vital roles in maintaining the healthy function of the body's immune system, preventing infection as well as having powerful anti-inflammatory, cardioprotective, neuroprotective, and anti-aging functions in the body. For example, magnesium deficiency alone and its widespread occurrence has been shown to be a primary driver in mitochondrial dysfunction, metabolic syndrome, depression, anxiety, cognitive impairment, dementia, attention deficit disorder, sleep disorders, immune dysfunction, high blood pressure, and cardiovascular disease. As a primary anti-inflammatory nutrient, magnesium reduces oxidative stress and inflammation by decreasing a number of pro-inflammatory cytokines including interleukin (IL)-1, the messenger cytokine IL-6, TNF-α, and C-reactive protein (CRP), which are known to play a key role in the health and function of the body's tissues. Along with the B-complex vitamins [e.g., thiamine (B1), riboflavin (B2), niacin (B3), pantothenate (B5), pyridoxine (B6), biotin (B7), folate (B9), cobalamin (B12)], and antioxidant vitamins and minerals: A, C, D, E, copper, iron, selenium, and zinc-magnesium serves important neuroprotective functions in the body—and acts as a vital “protective shield” against a wide range of environmental toxins and stress. Magnesium is only one nutrient deficiency prevalent in Western society. There are many more that further exacerbate this and other common deficiencies present (e.g., B-complex, copper, selenium, zinc, glycine, taurine).

Mitochondrial dysfunction (i.e. oxidative decay, mitochondrial damage), which is a major contributor to aging and a number of chronic disease states, is accelerated by many common micronutrient deficiencies. According to Director of the Cleveland Clinic Center for Functional Medicine, Dr. Mark Hyman, “when mitochondria are damaged, we suffer from low energy, fatigue, memory loss, pain, rapid aging, and more”—to name just a few symptoms of compromised mitochondrial function. Common factors that increase mitochondrial dysfunction and damage include: deficiencies in magnesium, copper, selenium, zinc, thiamine, riboflavin, niacin, pyridoxine, biotin, pantothenic acid, glycine, and taurine—and excess refined sugar, processed foods, toxin exposure, and lack of exercise. An optimum intake of micronutrients protects highly sensitive mitochondria from free radical injury (oxidative stress), promoting healthy mitochondrial function, and reversing mitochondrial dysfunction. Reestablishing healthy mitochondrial function has a beneficial effect on organs and tissues, and can dramatically enhance a person's overall health and well-being.

According to biochemist, Dr. Bruce N. Ames in his 2006 paper, Low micronutrient intake may accelerate the degenerative diseases of aging through allocation of scarce micronutrients by triage, “The triage theory provides a unifying rationale for why modest vitamin/mineral (V/M) deficiencies—insufficient to elicit overt symptoms of severe deficiency—might contribute significantly to the aging process and the diseases of aging. Briefly, the triage theory posits that a strategic rationing response has been selected through evolution, which ensures that when a moderate shortage of a V/M is encountered, the scarce V/M is preferentially retained by those V/M-dependent proteins/enzymes that are essential for survival and reproduction, such as proteins essential for early development and immediate survival (i.e., “survival proteins”).

Proteins and enzymes needed for maintaining long-term health by preventing insidious damage are starved for vitamins and minerals and become increasingly inactive, leading to an increase in diseases of aging. A major aspect of degenerative aging is that the damage is insidious and clinically not obvious because it accumulates slowly over time and is apparent only later in life. The connection to V/M and other nutrient shortages is categorically underappreciated by our current medical system. The average American diet coupled with the exposure to a wide range of nutrient-depleting antagonists and toxins (e.g., stress, heavy metals, medications) does not afford an optimal degree of nutrition or antioxidant support to prevent neurological, cardiovascular, and immunological deficits leading to disease.

A number of biologically active and essential organic molecules exist throughout the plant and animal kingdoms serving vital and protective functions. When we consume fruits, vegetables, and animal products we consume these organic molecules in varying amounts. These molecules include carbohydrates, fats, proteins, amino acids, vitamins, chelated minerals, organic acids, and various phytonutrients. In modern nutritional science, medicinal chemistry, and pharmacology these different classes of organic molecules have been well-studied and put to use in modern medicine to ultimately advance the health of society except for one.

Nutritional mineral chelates (i.e. chelated minerals) and the biologically active organic acids used in the transport of these minerals is one such area that has gone largely ignored by the mainstream science and medical communities. Organic mineral chelates—also fundamentally referred to as organic “counterions” in traditional chemistry consist of biologically essential molecules such as glycine (glycinates), malic acid (malates), and taurine (taurinates), which support vital cellular functions in both humans and animals, and enhance the benefits of the essential minerals they're delivering to the body.

For example, nutritional supplements containing malic acid (malate) as a chelate, not only deliver a well-absorbed, stable form of an essential mineral such as magnesium (e.g., magnesium malate) to the body, but also have the extra benefit that malic acid provides as a potent detoxifier of aluminum and as a direct fuel (energy) source in mitochondrial ATP production. Some of the vital cellular functions that these organic chelates support include: antioxidation (glycine, taurine), *glutathione biosynthesis and upregulation (glycine, taurine), detoxification (glycine, malate, taurine), neurotransmitter and receptor modulation (glycine, taurine), regulation of inflammation (glycine, taurine), and mitochondrial energy production (malate, taurine). * Reduced glutathione, often referred to as the “master antioxidant” for its critical free-radical neutralizing functions, is a prominent scavenging antioxidant within cells alongside ascorbate, d-alpha tocopherol, coenzyme Q10, glutathione peroxidase, superoxide dismutase, and catalase.

In addition to providing protection from nonspecific oxidant damage and acting as an electron donor for glutathione peroxidase and glutaredoxin, reduced glutathione reverses the pro-oxidative signaling mediated by hydrogen peroxide that plays a prominent pathogenic role in many health disorders. It is also utilized in the conjugation and excretion of xenobiotics. Cellular levels of this indispensable antioxidant have been shown to decline during the aging process—with much of this decline, the result of deficiencies in important micronutrients (e.g., vitamins, minerals, glycine) required to maintain optimal levels of intracellular glutathione.

We know that a variety of micronutrients are required to maximize and optimize tissue levels of glutathione, the body's master antioxidant. Glutathione serves as a primary free-radical scavenging, and heavy metal/xenobiotic detoxifying molecule. It's been said that with knowledge comes great power. In this case, the power is in optimizing an individual's health, vitality, and overall well-being, while simultaneously preventing a multitude of chronic diseases. Magnesium, copper, zinc, lithium, molybdenum, selenium, vitamins A, B-complex, C, D, E, taurine, and glycine play a role in maintaining optimal (and sufficient) tissue levels of glutathione for many biochemical and protective functions in the body.

Thus, what is needed is a broad-spectrum nutrient supplementation incorporating these elements in the proper (i.e. optimal) amounts-utilizing our latest understanding of nutrient optimization, nutritional biochemistry, and the “triage effect”—for the greatest benefit.

Cannabinoids and the neuroprotective B-vitamin, niacinamide, are known to have potent antioxidant and anti-inflammatory properties both partially dependent of, and independent of NMDA receptor inhibition, respectively. Cannabinoids exert their beneficial effects through their innate free-radical scavenging (antioxidant) activity, CB1 and CB2 receptor interaction, and epigenetic modulation whereas niacinamide (vitamin B3) exerts its influence through a number of mechanisms including: the inhibition of and quenching of mitochondrial ROS, the creation of antioxidant, reducing molecules nicotinamide adenine dinucleotide (NADH) and nicotinamide adenine dinucleotide phosphate (NADPH), supporting efficient energy metabolism, and the upregulation of endogenous, antioxidant enzyme systems.

Copper deficiency. The use of pharmaceutical drugs can deplete a variety of vital micronutrients (e.g., magnesium, vitamin D, folate). Trace mineral imbalances and deficiencies can also occur under a wide variety of conditions. One example is copper deficiency—an important mitochondrial nutrient-induced by the relatively common and excessive use of zinc supplements. Zinc supplementation increases the expression of the intestinal copper-binding protein, metallothionein, which binds copper and other metals making them unavailable for absorption.

It has been shown that both zinc deficiency and zinc excess can promote oxidative stress. Excess zinc supplementation as a result of taking multivitamins, individual zinc supplements (e.g., especially containing 30-50 mg elemental zinc per serving), and zinc lozenges (containing zinc without sufficient copper or devoid of copper) are some of the primary causes of copper deficiency. Moderate to high zinc supplementation alone can induce sufficient intestinal copper loss to promote a deficiency, which can be further intensified when combined with high-dose, vitamin C supplementation (e.g., >2,000 mg/d) and/or excess refined sugar consumption, which inhibit intestinal copper absorption and/or promote biliary/intestinal copper excretion. Other minerals commonly found in multivitamin preparations such as calcium, magnesium, manganese, and molybdenum also prevent copper absorption and/or encourage increased copper excretion.

To add further detail to the above, zinc supplementation can exacerbate a pre-existing low-level copper deficiency. Zinc supplementation is very common and is a contributing factor in promoting copper deficiency, along with excess vitamin C, refined sugar (sucrose), and fructose consumption. Vitamin C supplementation, especially in excess of 2,000 mg per day can exacerbate a pre-existing low-level copper deficiency. Vitamin C supplementation is very common and is a primary contributing factor in promoting copper deficiency, along with excess zinc, sucrose and fructose consumption. Copper deficiency negatively effects mitochondrial energy production, brain health and neurological function, the heart and cardiovascular system, the immune system and the protection against infections, thyroid function, and the health of bones and teeth.

Along with magnesium, selenium, and zinc-copper is an important trace element in the prevention of premature aging. Insufficient copper impairs cellular respiration (energy production), cognitive function, collagen and elastin production, and can contribute to bone loss and tooth degeneration (e.g., enamel loss). In short, copper deficiency is an under-recognized contributing factor in premature aging.

Copper deficiency is more common than presently recognized by the current healthcare system. As previously alluded to, soil depletion of essential trace minerals is widespread leading to decreased intake of vital, protective minerals such as copper necessary for a multitude of biological processes. In addition to soil depletion, a number of copper antagonists exist such as vitamin C, and divalent metals such as calcium, magnesium, zinc, manganese, and molybdenum, which compete for intestinal absorption of copper and/or promote its excretion, and are plentiful in multivitamins, antacids, and other dietary supplements (e.g., vitamin C tablets, capsules, or packets; zinc lozenges), which further increase the likelihood of developing a suboptimal copper level or overt copper deficiency.

Copper ions are required for cellular processes such as respiration, neurotransmitter biosynthesis, tissue growth and healing, collagen synthesis, and defense against oxidative stress and iron metabolism. A sufficient intracellular copper level must be maintained in order to preserve these important processes. Copper deficiency can lead to loss of the functions of enzymes, including cytochrome c oxidase, lysyl oxidase, dopamine-b-hydroxylase, superoxide dismutase, and ceruloplasmin that are required for numerous cellular processes. A lack of sufficient copper can result in: 1) fatigue, 2) low mood, 3) reduced cognitive function, 4) skin problems, 5) impaired collagen production, 6) accelerated aging (e.g., wrinkles, gray hair), 7) reduced restorative sleep (e.g., decreased melatonin secretion), and 8) joint stiffness and arthritis. Benefits of sufficient copper intake (tissue levels) include: 1) promotes healthy, youthful skin, 2) supports collagen production for improved skin and bone health, 3) promotes a healthy mood, and improves cognitive function, 4) promotes healthy energy levels, 5) supports healthy joint and muscle function, 6) promotes iron utilization (e.g., copper and iron are companion nutrients), and 7) improves sleep quality.

Glycine deficiency. Glycine is an example of a conditional vitamin because it is synthesized by humans and animals, but not in sufficient amounts, and thus must be consumed from the diet (or supplements)—for optimal health and optimal protective effects for healthy longevity and disease prevention. Glycine is required for the production of the body's “master antioxidant”, glutathione, plays a key role in regulating brain and nervous system function, and is an important building block for many chemical processes.

Glycine has been shown to be important in supporting optimal brain function, and the health and tissue function including: the skin, liver (glutathione, detoxification), cardiovascular system, bone, joints, and connective tissue, and in the prevention of a number of health issues including brain dysfunction, premature aging, metabolic syndrome, high blood pressure, and joint problems.

Regarding the health protective potential of supplemental glycine, in addition to its role as a precursor of glutathione, glycine is a substrate in the synthesis of porphyrins (heme), purines, creatine, sarcosine, and bile salts. Glycine comprises approximately one-third of the amino acids in collagen and elastin and thus serves as a primary component of connective tissues and the extracellular matrix. In addition, glycine has been shown to suppress protein glycation and the subsequent formation of advanced glycation end-products (AGEs)—proteins or lipids that become glycated as a result of exposure to sugars. In conjunction with its role in promoting glutathione biosynthesis and optimal glutathione levels in the body for cellular support and detoxification, in hepatocytes, glycine is metabolized to pyruvate, where pyruvate can function as a scavenging antioxidant for hydrogen peroxide.

The fact that collagen and elastin are rich in glycine suggests that optimal glycine nutrition might be beneficial for the health of connective tissues (including the cardiovascular system. Through its interaction with glycine and NMDA receptors, glycine has been shown to have a calming effect on the brain and nervous system. A 2012 study in the Journal of Pharmacological Sciences found that glycine supplementation prior to bedtime improved sleep quality in individuals with difficulty sleeping, though daytime administration did not cause drowsiness.

Glycine supplementation has been shown to have a beneficial effect in reversing metabolic syndrome. It provides protection from the adverse effects of a high-fructose diet, exerting favorable effects on insulin sensitivity, blood pressure, serum free fatty acids, and intraabdominal fat stores. Glycine supplementation was also associated with significant reductions and improvements in systolic blood pressure and plasma markers of oxidative stress.

Glycine supplementation has been shown to have an anti-inflammatory effect via glycine-gated chloride channels on the surface of macrophages, leukocytes, and Kupffer cells where glycine exerts a hyperpolarizing effect, inhibiting Ca²⁺ influx via voltage-sensitive Ca²⁺ channels, thus downregulating their proinflammatory activity. Glycine has also been shown to protect the livers of alcohol-fed rodents, which is believed in part due to a suppression of Kupffer cell activation. In addition, glycine supplementation has been shown to improve liver status in rat models of nonalcoholic steatohepatitis induced by poor diet (e.g., high-fat, high-sugar) or by methionine/choline deficiency. Glycine has also been shown to benefit rodent models of inflammatory arthritis.

Taurine deficiency. Taurine is another conditional vitamin because it is synthesized by humans and animals, but not in sufficient amounts. Thus, it must be consumed from the diet (or supplements)—for optimal health and protective effects for healthy longevity and disease prevention.

Taurine serves as a regulator of mitochondrial protein synthesis, enhancing electron transport chain activity, and protecting mitochondria against excessive superoxide generation. There is abundant evidence that taurine protects against free-radical generated oxidative stress, and has been shown to exert protective effects on vascular structure and function in several models of cardiovascular disease through its antioxidant and anti-inflammatory properties. Restoration of taurine levels restores respiratory chain activity, decreases superoxide anion production, and increases the synthesis of ATP. Taurine has been shown to be important in preventing numerous health issues including brain dysfunction, cardiovascular disease, diabetes, and mitochondrial diseases in large part due to its ability to reduce oxidative stress and inflammation (i.e. antioxidant and anti-inflammatory properties).

Taurine's positive effects on cardiovascular disease have been examined by numerous random controlled trials. Taurine supplementation has been shown to lower blood pressure, improve vascular function, and raise plasma hydrogen sulfide levels in prehypertension patients. Taurine consumption was the most significant factor associated with reduced risk of ischemic heart disease in two international epidemiological studies of CVD in 61 populations (25 countries; n=14,000): Japanese people in Okinawa had the highest taurine dietary intake and the lowest incidence of ischemic heart disease and longest lifespan. In contrast, Japanese immigrants in Brazil who eat little seafood, but more meat and salt, had a 17-y shorter lifespan as a consequence of a very high ischemic heart disease mortality. Other human clinical studies showed that taurine decreases platelet aggregation, serum cholesterol levels, LDL/triglyceride levels, and enhances cardiac function.

Taurine plays an important role in brain health and development, including such processes as neuronal proliferation, stem cell proliferation, and differentiation, and is not toxic in humans. It functions as a neuromodulator in the brain and nervous system where it inhibits the N-methyl-D-aspartate (NMDA) receptor, while activating the GABA- and glycine-insensitive chloride channel. Taurine is neuroprotective and is required for neural development, and the formation of new neurons (neurogenesis).

Diabetic remediation by taurine has been reviewed previously. Its supplementation remediates diabetic pathologies, including retinopathy, neuropathy, nephropathy, cardiopathy, atherosclerosis, altered platelet aggregation, and endothelial dysfunction. In patients with type 1 and type 2 diabetes the taurine transporter is up-regulated in mononuclear blood cells, indicating that increased levels of taurine are sought and required by the cell. In rats, taurine reduces oxidative stress caused by diabetes.

Taurine is essential for fetal development, because the human fetus is unable to synthesize taurine, which is provided by the mother. Taurine is required for organ development and protects against the development of type 2 diabetes. As such, taurine is also a survival vitamin, and is well-established as an important conditional vitamin for survival functions and for healthy longevity in both humans and experimental animals.

Because of taurine's extensive involvement in a number of chronic health issues that lead to a decline of overall health and well-being, it is considered a longevity nutrient. It is located in the cytosol and mitochondria, and is present in tissues at millimolar concentrations. Taurine is notably high in electrically excitable and secretory tissues and in platelets. A 70-kg (154 lb) human contains approximately 70 g of taurine. In humans and animals, diet is the primary source of taurine (a sulfur-containing molecule) with smaller amounts synthesized endogenously in the liver from the two sulfur-containing amino acids, methionine and cysteine. Dietary sources of taurine include milk, dairy, and grains containing on average less than 25 mg per serving, and richer sources such as fish, seaweed, eggs, and dark-meat poultry containing greater than 50 mg per serving. The average daily taurine intake for adult, non-vegetarians has been estimated between 40 and 400 mg, typically falling closer to the lower end of the range.

Taurine plays an important role in brain development, including neuronal proliferation, stem cell proliferation, and differentiation; it has no toxic effects in humans. It is a neuromodulator in the central nervous system: it activates the GABA- and glycine-insensitive chloride channel and it inhibits the N-methyl-D-aspartate receptor. It is also neuroprotective and has a role in neural development and neurogenesis.

Taurine reduces the oxidative stress generated by heavy metals, and assists with their removal from the body. When coadministered with DMSA or MiADMSA, taurine helped to further reduce total body burden of arsenic and lead.

Therapeutic use of malic acid. Malic acids serves as a Citric Acid Cycle (Kreb's Cycle) intermediate and is essential to life. Malic acid provides 2.39 nutritional calories (Calories) of energy per gram during digestion. The salts and esters of malic acid are known as malates. The word malic is derived from the Latin malu, meaning apple. Malic acid was first isolated from the juice of apples by Carl W. Scheele in the late 18^{th} Century (1785). Unripe, sour apples contain high proportions of the acid with the amount decreasing with increasing fruit ripeness. Malic acid is found naturally in fruits and a variety of vegetables. It is the primary acid found in many fruits including apricots, blackberries, blueberries, cherries, grapes, peaches, pears, and plums and is present in lower concentrations in other fruits, such as citrus. In citrus fruits, organic farming practices have been shown to produce higher levels of malic acid than their conventionally grown counterparts.

Malic acid (malate) is a natural aluminum chelating and detoxifying agent found throughout nature. Plants have been shown to up-regulate the production and secretion of organic acids such as citric acid, malic acid, and oxalic acid in response to aluminum and other toxic stressors. Once secreted, these acids, and more specifically their associated anions chelate Al³⁺ ions, protecting the secreting plant from aluminum toxicity. Chelation studies in animals have shown particular efficacy for both citric and malic acid in mobilizing and increasing fecal and urinary excretion of aluminum and other toxic metals.

Micronutrients function as vital “protective shields”. Micronutrients function as powerful antioxidants and/or precursors or modulators of the body's antioxidant defense systems (e.g., glutathione, superoxide dismutase); as such, they function as vital “protective shields” protecting vital cellular structures (e.g., mitochondria, cell membranes) in humans, animals, and all forms of life. Micronutrient deficiencies can result in DNA damage, which may ultimately lead to such degenerative states such as cancer. A deficiency of one or more of the following nutrients: vitamins C, E, B12, B6, niacin, folic acid, iron or zinc appears to mimic radiation by causing single- and double-strand DNA breaks, oxidative lesions or both. Micronutrient deficiencies may thus contribute to the increase of cancer incidence in the quarter of the population that cat the fewest fruits and vegetables, as compared with the quarter who have the highest intake. Although 5-9 portions of fruit and vegetables a day are advised, 80% of American children and adolescents and 68% of adults do not eat five portions daily.

Insufficient zinc causes oxidative DNA damage, inactivation of copper/zinc superoxide dismutase, inactivation of tumour suppressor protein p53—a zinc protein—and inactivation of oxidative DNA repair in cultured human cells, and these effects can multiply to cause severe genetic damage. Zinc deficiency is associated with cancer in both humans and rodent models but 10% of the US population ingest less than 50% of the RDA of zinc. Iron deficiency has also been shown to cause oxidative damage to mitochondria and mitochondrial DNA in rats. Among women of menstruating age in the USA, 25% ingest less than 50% of the RDA of iron. Due to the availability and heavy consumption of highly refined and processed foods (e.g., white flour baked goods, sodas), the poor are most affected by nutritional deficiencies as they have the lowest intake of these essential minerals.

Micronutrient deficiencies have a negative effect on mitochondrial metabolism and thus accelerate cellular aging. The biosynthesis of the heme protein occurs primarily in mitochondria and interfering with this process causes specific loss of heme-a—a component of mitochondrial complex IV—with a resulting release in oxidants and an increase in oxidative stress. Thus, iron deficiency promotes mitochondrial decay and the release of oxidants, seemingly through the lack of heme-a.

Deficiencies in copper and pantothenate impair mitochondrial metabolism through decreasing levels of complex IV, whereas biotin deficiency causes defects in this same complex thereby inducing oxidant leakage. Zinc deficiency causes mitochondrial impairment due to the inactivation of 8-aminolevulinate dehydratase, a zinc-containing enzyme for heme biosynthesis, resulting in increased release of oxidants. The consequences of these various micronutrient deficiencies are likely to be accelerated aging and poor overall health.

An increased intake of micronutrients constituting in-effect that of a “metabolic tune-up” can have major health benefits for the majority of Western society who are deficient in one or more of the aforementioned nutrients. Sufficient and optimum intake of essential micronutrients are important for optimal health, well-being, and longevity while preventing chronic disease. Micronutrient deficient, poor, and unbalanced diets are the largest contributor to ill health.

For more rapid onset, and maximum therapeutic benefit and efficacy-lipophilic, uncharged, poorly-ionized, mineral chelates such as those bound to glycinate are vehicles (carriers) for efficient, mineral delivery and absorption (i.e. high-bioavailability). Small molecule, mineral chelates such as those bound to glycinate (e.g., bisglycinates of Ca, Mg, Cu, Mn, Mo, Se<250 Da) are absorbed via hydrophilic, paracellular transport and lipophilic, transcellular transport, and/or through transcellular, carrier-mediated orotate or glycine transporters.

A profound deficit in current nutritional supplements available to the public. As stated previously, the vast majority of food-form (e.g., soft chew, gummy) multivitamins currently available (FIGS. 1-5) are often poorly balanced, lacking (e.g., containing 25% or less of the RDA of thiamine, riboflavin, niacin, zinc, copper, manganese, selenium), or completely deficient in specific neuroprotective, cardioprotective, immunity enhancing, mitochondrial nutrients.

Many popular gummy products marketed as “complete” (e.g., VitaFusion MultiVites, Vitafusion Men's and Women Multivitamins; SmartyPants Multivitamins; Nature's Way Alive Men's/Women's Multivitamins) are often completely missing key nutritional elements such as thiamine, riboflavin, niacin, copper, selenium, and zinc. From a medical and health perspective they may be marketed in a manner that is misleading and disserves the consumer (FIGS. 1-5) who believes they are receiving “complete” nutritional support for a wide-spectrum of key nutrients. These may include supporting healthy brain and nervous system function, a healthy heart and cardiovascular function, a strong immune system, cancer prevention, and the secondary antioxidant benefits of many nutrients (e.g., thiamine, riboflavin, copper, selenium, glycine, taurine) in reducing oxidative stress, unabated free-radical generation, and inflammation-known to be primary drivers in chronic disease and premature aging.

Accordingly, what is needed is a highly bioavailable, optimally dosed and balanced, multi-nutrient, antioxidant, anti-inflammatory preparation. Preferably it would contain many if not most of the above mentioned micronutrients for the prevention and treatment of a wide range of health conditions where oxidative stress and mitochondrial dysfunction plays a primary role. While many attempts have been made to address these needs, compositions that fulfill these objectives above remains elusive. Also needed is a neuroprotective, antioxidant, anti-inflammatory preparation for the prevention and treatment of a wide variety of primary and secondary health issues (e.g., cognitive decline, poor focus or concentration, sensitivity to stress, sleep disturbances) involving the brain and nervous system that is highly efficacious, non-toxic, and possesses high-bioavailability (easily absorbable into the systemic circulation), while passing readily across the blood-brain barrier. With its potent antioxidant, anti-inflammatory activity, this novel, hemp oil/niacinamide neuro- and cellular-protectant preparation, as described—in addition to its broad-spectrum neuroprotective activity—can be employed for a wide range of skin conditions (e.g., acne, eczema, dermatitis, psoriasis) known to have chronic inflammation, as a common, underlying component.

Disclosed is a preparation from common solvent extraction methods (e.g., ethanol, supercritical CO₂, butane) of hemp, including niacinamide for oral absorption, vaporization (inhalation), and topical delivery.

In some embodiments, a blend of synergistic nutrients is coupled to the broad- or full-spectrum hemp oil extract, niacinamide base for additional potency and therapeutic benefits.

In some embodiments, the neuroprotective preparation comprises vitamin B3 (e.g., niacinamide) and a hemp oil extract containing purified, cannabigerol, and a full-spectrum of naturally occurring cannabinoids obtained from the hemp plant. In some embodiments, the neuroprotective preparation comprises vitamin B3 (e.g., niacinamide) and a hemp oil extract containing purified, beta-caryophyllene, and a full-spectrum of naturally occurring cannabinoids obtained from the hemp plant. In some embodiments, the neuroprotective preparation comprises vitamin B3 (e.g., niacinamide) and a hemp oil extract containing purified, cannabinol, and a full-spectrum of naturally occurring cannabinoids obtained from the hemp plant.

In some embodiments, the hemp oil extract is derived from organically grown hemp. In some embodiments, the hemp extract is derived from conventionally grown hemp. In some embodiments, the hemp extract is prepared via an ethanolic extraction process. In some embodiments, the hemp extract is prepared via a super-critical, CO₂ extraction process. In some embodiments, the hemp extract is prepared using butane, hexane, naphthalene, coconut oil, olive oil, or other oils or hydrocarbon solvents.

In some embodiments, the neuroprotective preparation further comprises a hemp extract (i.e. hemp oil) containing a full spectrum of naturally occurring phytochemicals (e.g., terpenes, phenolics, cannabinoids) obtained from the hemp plant. In some embodiments, the hemp oil is derived from organically grown hemp. In some embodiments, the hemp oil is derived from conventionally grown hemp.

This invention relates to cognitive-enhancing, stress-reducing, antioxidant, anti-inflammatory preparations containing hemp oil and the basic (amide), non-flushing form of vitamin B3, niacinamide.

Disclosed is a broad- or full-spectrum cannabinoid preparation from hemp oil, coupled with niacinamide in a highly efficacious formula for oral absorption, vaporization (inhalation), or topical delivery.

In some embodiments, the neuroprotective preparation comprises vitamin B3 in the form of nicotinamide riboside, either alone or in addition to niacinamide.

In some embodiments, the amount of hemp oil is between 0.5 and 500 milligrams per serving.

In some embodiments, the amount of cannabidiol (CBD) is between 0.25 and 100 milligrams per serving.

In some embodiments, the amount of niacinamide is between 0.5 and 250 milligrams per serving.

In some embodiments, the amount of nicotinamide riboside is between 0.5 and 250 milligrams per serving.

### BRIEF DESCRIPTION OF THE DRAWINGS

FIG. 1 is a picture and the supplement facts of Vitafusion MultiVites-2 gummies per serving which completely lacks vitamin B1 (thiamine), B2 (riboflavin), magnesium, zinc, copper, manganese, and selenium;

FIG. 2 is a picture and the supplement facts of Vitafusion Men's Gummy Vitamins, Multivitamin-2 gummies per serving which completely lacks vitamin B1 (thiamine), B2 (riboflavin), B3 (niacin), magnesium, copper, manganese, and selenium;

FIG. 3 is a picture and the supplement facts of Vitafusion Women's Gummy Vitamins, Multivitamin-2 gummies per serving which contains less than 25% of the RDA for zinc, and completely lacks vitamin B1 (thiamine), B2 (riboflavin), B3 (niacin), magnesium, copper, manganese, and selenium; and

FIG. 4 is a picture and the supplement facts of Smarty Pants Adult Formula, Daily Gummy Multivitamin-6 gummies per serving which contains less than 25% of the RDA for vitamin B1 (thiamine), B2 (riboflavin), and completely lacks vitamin B3 (niacin), magnesium, copper, manganese, and selenium.

FIG. 5 is a picture and the supplement facts of Olly The Perfect Men's Multi. Gummy Multivitamin-2 gummies per serving which contains less 25% of the RDA for vitamin B1 (thiamine) and B2 (riboflavin), 50% of the RDA for vitamin B3 (niacin), and 250% of the RDA for vitamin B6. Contains no magnesium, copper, manganese, and only 33% of the RDA for zinc.

### DETAILED DESCRIPTIONS OF THE INVENTION

The drawings are for the purpose of describing selected versions of the present invention and are not intended to limit the scope of the present invention.

The embodiments herein relate to novel, multi-nutrient, antioxidant preparations containing organic mineral chelates (e.g., glycinates, malates, taurinates) for optimizing neurological, cardiovascular, and immune system function for broad-spectrum mitochondrial support, and multisystem protective effects. The novel, multi-nutrient preparations presented herein possess broad-spectrum antioxidant, anti-inflammatory activity, which may confer superior neuroprotective, cardioprotective, immunoprotective, and anti-aging benefits. This formulation represents the culmination of over 30 years of research and clinical study of nutritional biochemistry, orthomolecular nutrition, general nutrition, nutrient therapeutics, and functional medicine.

An optimal, therapeutic intervention to prevent and/or treat unabated oxidative stress and associated mitochondrial injury (damage) would ideally create a microenvironment wherein oxidative stress is minimized or reduced. Additionally, the therapeutic must be convenient for use in humans and animals—and easy to take—making a “soft-chew”, “gummy”, lozenge, chewable tablet, water soluble powder in packet form, “food-form,” partially orally-absorbable preparation greatly advantageous. Tablet and capsule forms of the preparation (formulation) will also be provided and will provide similar benefits with a slower onset of therapeutic effects.

The embodiments contained herein relate to preparations containing a full-spectrum of antioxidant nutrients (e.g., glycine, taurine, magnesium, selenium) and/or nutraceuticals (e.g., coenzyme Q10, alpha lipoic acid) and/or plant-based antioxidants (e.g., terpenes, polyphenolics) working in perfect biological synergy at optimal therapeutic doses for exceptional mitochondrial protection, function, and repair.

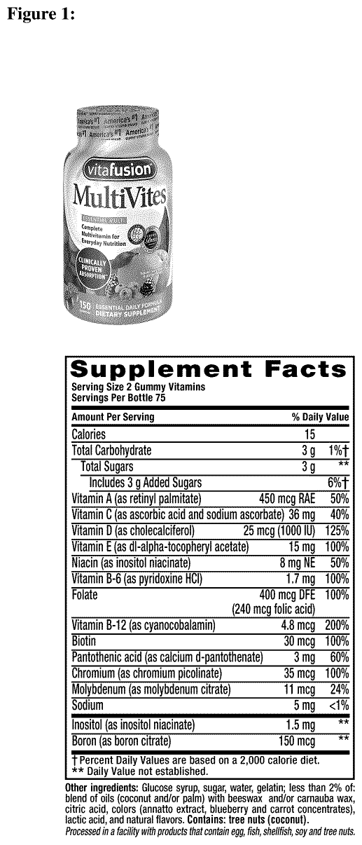

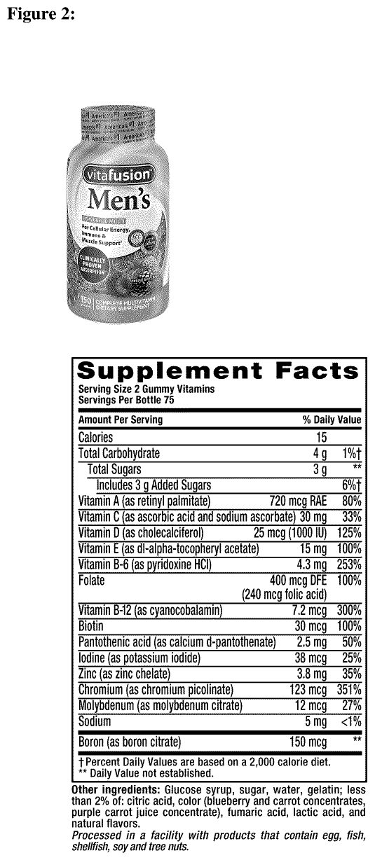

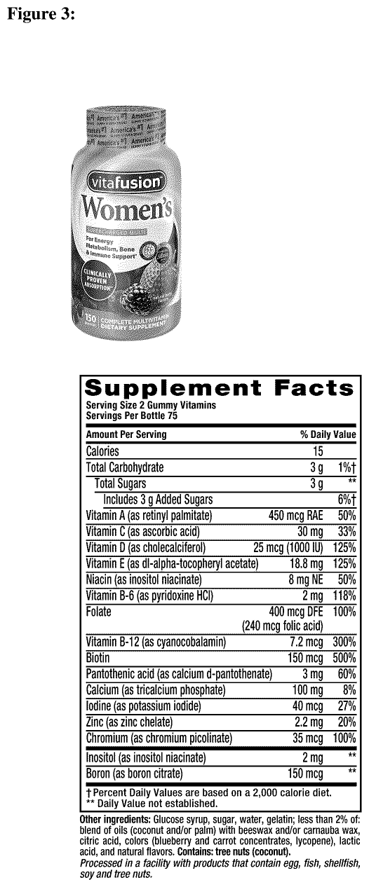

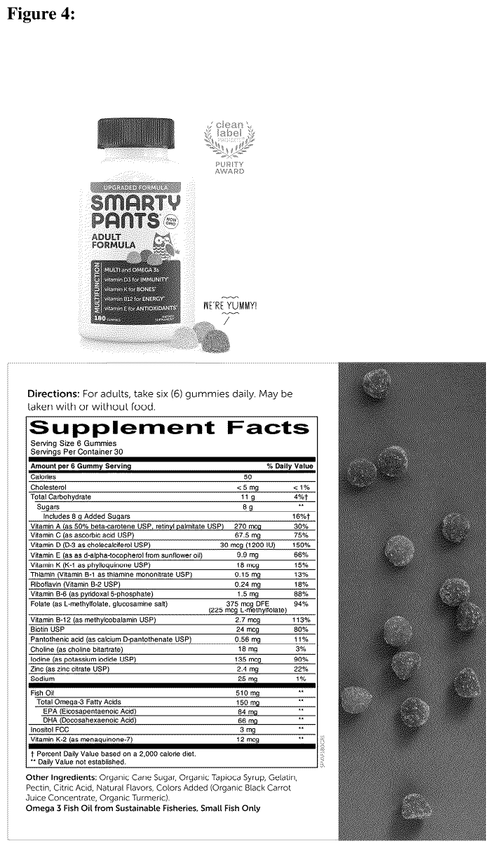

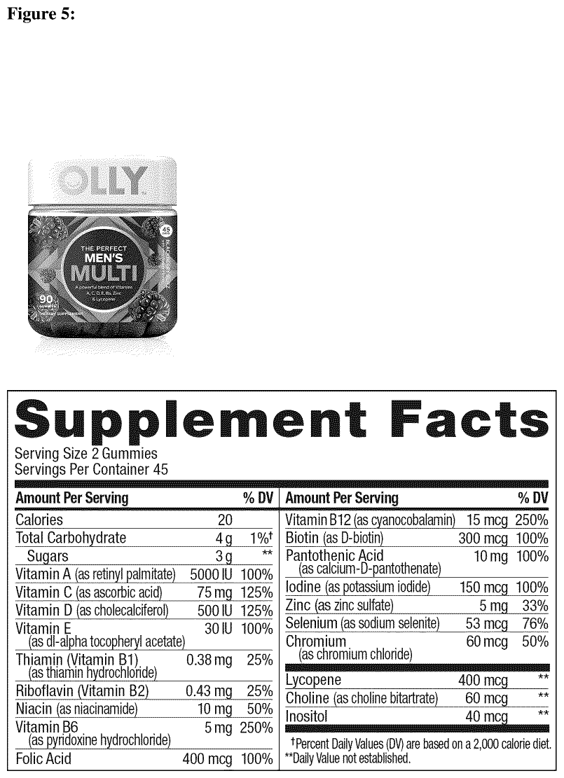

The average American diet coupled with the exposure to a wide range of nutrient-depleting antagonists and toxins (e.g., stress, heavy metals, medications) does not afford an optimal degree of nutrition or antioxidant support to prevent mitochondrial, neurological, cardiovascular, and immunological deficits leading to disease. In addition, the majority of multivitamins on the market are subpar at best, especially food-form supplements (e.g., soft chew, gummy). They are often poorly balanced, deficient, or completely lacking in specific neuroprotective, cardioprotective, immune supportive, mitochondrial nutrients (FIGS. 1-5). Embodiments of the present disclosure consist of efficacious, mitochondrial supportive, cellular rejuvenating, anti-aging, broad-spectrum nutrient preparations that address and correct these deficits for consumer benefit.

In addition, the embodiments herein also relate to antioxidant, mitochondrial supportive preparations of hemp oil containing a full-spectrum of beneficial, nontoxic, phytochemicals in combination with highly-bioavailable, lipophilic, mineral chelates (e.g., glycinates) of magnesium and other essential minerals—in a blend for highly-efficient, oral (e.g., gingival, buccal absorption) and topical delivery, and methods of use in humans and animals.

An optimal, therapeutic neuroprotective, cardioprotective, immunoprotective (immunosupportive) intervention to prevent and/or treat unabated oxidative stress and associated mitochondrial injury would ideally create a microenvironment wherein oxidative stress and mitochondrial function and integrity is improved and preserved. Additionally, preventative therapy must be convenient for use and easy to take. A soft chew, gummy, chewable tablet, lozenge, quick-dissolve tablet, “chewing gum”, liquid spray, liquid beverage, liquid “shot”, tincture, “food-form,” or topical application formulations are advantageous.

The, multi-nutrient, antioxidant preparation, in some embodiments, is a commercially available blend of vitamins, minerals, and other antioxidant nutrients (e.g., glycine, taurine, nutraceuticals, phytochemicals) combined to improve mitochondrial function, reduce oxidative stress, and promote healthy neurological, cardiovascular and immunological function with optimal anti-aging support. In some embodiments, the preparation comprises (per serving; chewable tablet, soft chew, gummy, lozenge, tablet, capsule, or packet): glycine, 100-5,000 mg; taurine, 100-2,000 mg; malic acid, 100-2,000 mg; vitamin A as retinol, retinyl palmitate, retinyl acetate, or beta-carotene, 250-10,000 iu; vitamin B1, 0.25-100 mg; vitamin B2, 0.25-100 mg; vitamin B3, 5-250 mg; vitamin B5, 5-1,000 mg; vitamin B6, 0.25-100 mg; vitamin B12, 5-5,000 mcg; biotin, 5-1,000 mcg; PABA, 0.50-100 mg; vitamin C, 50-5,000 mg; vitamin D2 or D3, 400-10,000 IU; natural vitamin E (tocopherols and/or tocotrienols), 5-1,000 iu; boron (glycinate), 0.1-10 mg; copper (glycinate), 0.1-5 mg; zinc (glycinate), 2-50 mg; manganese (glycinate), 0.5-5 mg; selenium (e.g., glycinate), 10-400 mcg; molybdenum (glycinate), 10-400 mcg; and chromium (nicotinate glycinate or polynicotinate), 10-1,000 mcg.

The foregoing base formulation is by way of example only; other formulations of the, multi-nutrient, antioxidant, mitochondrial supportive preparation are contemplated by the present disclosure. In some cases, the formulation comprises one or more additional components such as, for example, coenzyme Q10 as ubiquinone or ubiquinol, 2.5-1,000 mg; alpha lipoic acid, 2.5-1,000 mg; kelp, 5-200 mg; Extramel French melon extract, 0.5-10 mg, AuroraBlue Alaskan Blueberry Concentrate or Extract, 10-2,000 mg; hemp oil extract, 1-1,000 mg supplying 0.5-100 mg of cannabidiol (CBD) and other cannabinoids, terpenes, and phenolics. Additionally, it is anticipated that as scientific investigation generates additional data regarding the role of the various compounds comprising the preparation are generated, the composition of the preparation will change accordingly.

The form of nutrient delivery in some embodiments is a highly specialized mucoadhesive delivery system, which facilitates oral bioavailability through mucosal absorption from within the oral cavity. Some non-limiting examples of such excipient compounds include xylitol, erythritol, allulose, cellulose, palm oil, coconut oil, silicon dioxide, citric acid, malic acid, organic stevia (leaf) extract, monk fruit extract, and natural flavors. Other such compounds as are known in the art of oral nutrient and/or drug absorption and delivery systems may be used. In these and some other embodiments, the mitochondrial preparation is taken as a tablet, capsule, powder packet, liquid, soft chew, gummy, chewable wafer, quick-dissolve tablet, or lozenge placed in the mouth. In some embodiments, the lozenge is held under the tongue or placed in a buccal recess and allowed to dissolve. In some embodiments, the soft chew is chewed, allowing exposure of the vitamins, minerals, and other nutrients to the microvascular tissue (e.g., gingival, buccal) of the oral cavity for efficient absorption of the nutrients, and swallowed.

Topical creams, lotions, salves, balms, “patches”, and other external methods of application could also be employed to deliver the antioxidant, mitochondrial preparation consisting of synergistic, antioxidants nutrients (e.g., vitamins, minerals, nutraceuticals) to the skin. Hence, in some embodiments, the novel, antioxidant mitochondrial supportive preparations, as described herein, could also be employed for a wide range of skin conditions (e.g., acne, eczema, dermatitis, psoriasis) known to have excess oxidative stress and chronic inflammation, as a common, underlying component. Cannabinoids and the essential mineral magnesium are known to have antioxidant and anti-inflammatory properties both independent of, and dependent on NMDA receptor inhibition, respectively. Cannabinoids exert their beneficial effects through their innate free-radical scavenging (antioxidant) activity, cannabinoid receptor activation, and epigenetic modulation whereas magnesium exerts its influence through NMDA receptor inhibition, and multi-modal, synergistic interactions with a wide-variety of vitamins (e.g., vitamin D, B-complex), minerals (e.g., lithium, zinc), and the upregulation of endogenous, antioxidant enzyme systems.

In view of the above, some embodiments relate to mitochondrial supportive (nourishing) and protective, antioxidant, anti-inflammatory preparations containing hemp oil with a broad-spectrum of beneficial, antioxidant phytochemicals and highly bioavailable, magnesium coupled with selected, synergistic vitamins, trace elements, and/or nutraceuticals. Other embodiments relate to hemp oil—containing a broad-spectrum of beneficial terpenes, phenolics, and cannabinoids—in combination with highly bioavailable magnesium enhanced with additional synergistic antioxidant nutrients and/or nutraceuticals for neuroprotective, cardioprotective, and immunoprotection for a wide-variety of health-related and skin benefits.

In some embodiments, the mitochondrial supportive preparation further comprises a hemp extract (i.e., hemp oil) containing a full spectrum of naturally occurring phytochemicals (e.g., terpenes, phenolics, cannabinoids) obtained from the hemp plant. In some embodiments, the hemp oil is derived from organically grown hemp. In some embodiments, the hemp oil is derived from conventionally grown hemp.

In some embodiments, the mitochondrial support preparation further comprises an inorganic counter-ion carrying a variety of essential minerals, such as carbonate, chloride, oxide, or sulfate. In some embodiments, the counter-ion is an organic chelating compound chosen from the group consisting of acetic acid, succinic acid, ascorbic acid, aspartic acid, threonic acid, lysinic acid, malic acid, tauric acid, citric acid, and gluconic acid. In some embodiments, the chelating compound is a salt of orotic acid. In some embodiments, the chelate is a salt of glycine.

In some embodiments, the amount of glycine is between about 100 and 5,000 milligrams per serving.

In some embodiments, the amount of taurine is between about 100 and 2,000 milligrams per serving.

In some embodiments, the amount of malic acid is between about 100 and 2,000 milligrams per serving.

In some embodiments, the amount of cannabidiol (CBD) is between about 0.50 and 100 milligrams per serving.

In some embodiments, the amount of elemental magnesium is between about 5 and 400 milligrams per serving.

In some embodiments, the amount of elemental copper is between about 0.1 and 5 milligrams per serving.

In some embodiments, the amount of elemental zinc is between about 2.0 and 50 milligrams per serving.

In some embodiments, the amount of elemental selenium is between about 5 and 400 micrograms per serving.

In some embodiments, the amount of elemental boron is between about 0.10 and 10 milligrams per serving.

In some embodiments, the amount of elemental chromium is between about 5 mcg and 1,000 micrograms per serving.

In some embodiments, the amount of iodine is between about 50 mcg and 500 mcg per serving.

In some embodiments, the amount of kelp is between about 5 mg and 200 mg per serving.

In some embodiments, the amount of elemental manganese is between about 0.10 and 10 milligrams per serving.

In some embodiments, the amount of elemental molybdenum is between about 5 mcg and 400 micrograms per serving.

In some embodiments, the amount of thiamine is between about 0.25 mg and 100 milligrams per serving.

In some embodiments, the amount of riboflavin is between about 0.25 mg and 100 milligrams per serving.

In some embodiments, the amount of niacin is between about 5 mg and 250 milligrams per serving.

In some embodiments, the amount of pantothenate is between about 5 mg and 200 milligrams per serving.

In some embodiments, the amount of pyridoxine is between about 0.25 mg and 100 milligrams per serving.

In some embodiments, the amount of cobalamin is between about 3 mcg and 5,000 micrograms per serving.

In some embodiments, the amount of PABA is between about 0.1 mg and 100 milligrams per serving.

In some embodiments, the amount of vitamin A is between about 250 iu and 10,000 iu per serving.

In some embodiments, the amount of vitamin C is between about 10 mg and 2,000 milligrams per serving.

In some embodiments, the amount of vitamin D is between about 50 iu and 5,000 iu per serving.

In some embodiments, the amount of vitamin E is between about 5 iu and 1,000 iu per serving.

In some embodiments, the amount of vitamin K2 (Mk-7) is between about 5 iu and 500 mcg per serving.

In some embodiments, the amount of coenzyme Q10 is between about 2.0 mg and 1,000 milligrams per serving.

In some embodiments, the amount of alpha lipoic acid is between about 2.0 mg and 1,000 milligrams per serving.

In some embodiments, the amount of AuroraBlue Alaskan Blueberry Concentrate is between about 5 and 5,000 milligrams per serving.

In some embodiments, the amount of Extramel French Melon Extract is between about 0.5 and 20 milligrams per serving.

As discussed above, the disclosed invention relates to preparations of hemp oil containing a full-spectrum of naturally-occurring phytochemicals (e.g., terpenes, phenolics, cannabinoids) obtained from the hemp plant in combination with niacinamide for oral, topical, and portable electronic vaporization delivery as a general antioxidant, anti-inflammatory, central nervous system neuroprotectant and neurotrophic, and methods of use.

A wide range of primary central neurological diseases and secondary conditions manifest cognitive, memory, motor, and sensory impairment as primary and debilitating symptoms. A few non-limiting examples of such diseases and conditions are listed herein above and include amyotrophic lateral sclerosis (“ALD”), Parkinson's Disease (“PD”), Alzheimer's Disease (“AD”), post-traumatic stress disorder (“PTSD”), attention deficit hyperactivity disorder (“ADHD”), depression/anxiety, and tinnitus. Examples of secondary conditions include traumatic brain injury (“TBI”), chronic post-traumatic or post-surgical neuropathic pain, acute or chronic exposure to certain toxins, for example mercury, lead, cadmium, arsenic, polychlorinated biphenyls, and ethanol; and cerebral ischemia.

Phytochemicals from hemp oil (e.g., cannabinoids, terpenes, phenolics) and niacinamide exert a myriad of antioxidant, anti-inflammatory benefits on a number of body systems including the brain and nervous system, liver, kidneys, and skin—promoting improved mood and cognitive function, stress reduction, decreased pain, and a wide-variety of health benefits.

The cannabinoids, terpenes, and phenolics in hemp oil exert a portion of their neuroprotective, antioxidant, anti-inflammatory effects through inhibition of the N-methyl-D-aspartate receptor (“NMDA”). NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

In addition to its use in a preventative capacity, the hemp oil/niacinamide therapeutic possesses a number of immediate benefits to the consumer including: decreased inflammation, decreased pain and tension, a calming/relaxing effect on the brain and body, and improved energy, mood, and cognitive function (e.g., memory, focus, concentration). By reducing neural and general inflammation in the body, the therapeutic helps to relieve tension, and promote restful, restorative sleep.

Niacin (also known as vitamin B3) includes two biologically active forms, nicotinic acid and nicotinamide (niacinamide), which give rise to the coenzymes, nicotinamide adenine dinucleotide (NAD) and nicotinamide adenine dinucleotide phosphate (NADP). NAD and NADP are coenzymes required for oxidative reactions important in energy production and non-redox signaling pathways regulating biological functions such as gene expression, cell cycle progression, DNA repair and cell death. The ratio of NAD and NADP play different metabolic roles in the cytosol: the NADH/NAD+ ratio is small (˜8×10⁻⁴), thus favoring oxidative catabolism, whereas the NADPH/NADP+ ratio is higher (˜75), thus providing a strongly reducing environment for biosynthetic reactions. NAD+ is an essential cofactor of enzymes involved in various signaling pathways that are important in cellular functions such as cellular energy metabolism, differentiation, and stress resistance. Niacinamide is considered to be the main NAD+ precursor in mammals and is involved in the coordination and regulation of a diversity of cellular responses, including proliferation, differentiation, and apoptosis, through the regulation of a wide range of enzymatic activities. As a precursor of NAD+, NADH, NADP+, and NADPH, niacinamide plays an integral role in cellular metabolism, cellular proliferation and the repair of damaged cells.

In the brain and nervous system, vitamin B3 (e.g., niacinamide) serves a vital role in neuroprotection, neurogenesis, neuronal development, and neuron survival (see Table 1). Like other neurotrophic vitamins and minerals (e.g., ascorbic acid, zinc) whose intake is often suboptimal or deficient due to a wide variety of factors (e.g., nutrient poor diets, excess refined sugar, increased requirements due to stress, medication induced deficiencies)—niacinamide in sufficient or optimal amounts promotes the formation of new neurons (i.e. neurogenesis) by promoting stem cell proliferation, stem cell survival, and increasing the rate of differentiation of embryonic stem cells or neural progenitors into post-mitotic neurons. Due to its importance in energy metabolism, and biosynthetic reactions essential for healthy brain function, niacinamide has been demonstrated to move freely in and out of the brain.

TABLE 1
Main findings on the role of niacin in neurodegeneration

| | Effector | Main Findings |
|---|---|---|
| Alzheimer's | Niacin | Inverse association between |
| disease | | AD and dietary niacin intakes. |
| | NAD⁺ | High brain levels restore |
| | | mitochondrial function and |
| | | antagonize cognitive decline. |
| | Nam/Nam | Protect against Aβ-induced |
| | mononucleotide | neurotoxicity via reduction |
| | | of App and PSEN-1 expression |
| | | and ROS levels. |
| | Nam riboside | Reduces DNA damage, neuro- |
| | | inflammation and cell death |
| | | of hippocampal neurons. |
| | SIRT1 | Supports the non-amyloidogenic |
| | | pathway of AD. |
| | | Lessens AD neuroinflammation, |
| | | oxidative stress and |
| | | mitochondrial dysfunction. |
| | NMNAT1-3 | Protects against axon |
| | | degeneration via reduction |
| | | of nicotinamide mononucleotide |
| | | levels and SIRT1 activation. |
| | NMNAT2 | Activity downregulated prior |
| | | to neurodegeneration; |
| | | restoration of activity is |
| | | neuroprotective against |
| | | tauopathy. |
| | | Low gene expression in AD |
| | | patients. |
| Parkinson's | Niacin | Increased intake enhances |
| disease | | striatal dopamine synthesis |
| | | and restores optimal NAD⁺/ |
| | | NADH ratio. |
| | | High levels sequester transition |
| | | metal ions. |
| | | Low doses impact macrophage |
| | | polarization from M1 (pro- |
| | | inflammatory) to M2 (anti- |
| | | inflammatory) profile. |
| | NAD⁺ | Decreased levels in PD patients. |
| | NADPH | Inhibits MPTP⁺-induced |
| | | oxidative stress and glia- |
| | | mediated neuroinflammation. |
| | NNMT | High levels in the cerebrospinal |
| | | fluid and midbrain dopamine |
| | | neurons of PD patients. |
| | | High activity associated with |
| | | low activity of mitochondrial |
| | | complex 1; it counteracts the |
| | | MPP⁺-dependent toxicity |
| | | on mitochondrial complex 1 and |
| | | activates neuronal autophagy. |
| | | Induces neurite branching, |
| | | synaptophysin expression and |
| | | dopamine release. |
| Huntington's | NAD | Low levels correlate with disease |
| disease | | progression in DrosophilaHD model. |
| | Nam | Protects against the toxicity of |
| | | polyQ proteins in DrosophilaHD |
| | | models. |
| | | Restores BDNF protein levels, |
| | | increases acetylated PGC-1α, |
| | | improves motor deficits. |
| | | Prevents motor abnormality via |
| | | PARP-1-dependent inhibition of |
| | | neuronal death and oxidative |
| | | stress. |
| | SIRT1 | Rescues neurons from mutant |
| | | huntingtin toxicity. |
| | | Ameliorates pathological |
| | | mechanisms underlying disease |
| | | onset. |

Cannabinoids and vitamin B3 (e.g., nicotinic acid, nicotinamide, niacinamide) are known to have antioxidant and anti-inflammatory properties both independent of, and dependent on NMDA receptor inhibition, respectively. By definition, antioxidants possess anti-inflammatory properties. Cannabinoids exert their beneficial physiological effects through a myriad of activities including antioxidation, CB1 and CB2 receptor interaction, and epigenetic modulation whereas niacinamide (vitamin B3) exerts its influence through its innate antioxidant activity, regeneration of endogenous antioxidants such as glutathione, and the support of numerous biochemical pathways involved in energy production, biosynthetic processes, and cellular repair. Evidence from both in vitro and in vivo studies demonstrate that niacinamide possesses potent antioxidant properties, and niacinamide deficiency contributes to increased oxidative stress and inflammation.

Preventative therapy or treatment must be convenient for use making a vaporization delivery method such as provided by a portable electronic vaporization device (e.g., vape pen) greatly advantageous. Given the similar boiling point temperatures for hemp oil and niacinamide, and their lack of harmful degradation byproducts, makes this combination especially well-suited for vaporization delivery. For example when heated to vaporization, the cannabinoids in hemp oil undergo a beneficial “activation process”, chemically referred to as decarboxylation (loss of a carboxyl group), and niacinamide like its chemical cousin nicotine with their pyridine rings, remain largely intact when exposed to temperatures that approach or exceed vaporization. A study published in the journal Chemical Research in Toxicology found that nicotine vaporized using e-cigarette delivery produced no detectable levels of pyridine (a nicotine breakdown product) in the aerosolized vapor. Of the 150 analytes measured in the e-cigarette aerosol, 104 were not detected, and 9 out of the 25 aerosol analytes detected were too low to be quantified. The authors concluded, “thus, the aerosol from the e-cigarette is compositionally less complex than cigarette smoke and contains significantly lower levels of toxicants. These data demonstrate that e-cigarettes can be developed that offer the potential for substantially reduced exposure to cigarette toxicants.”

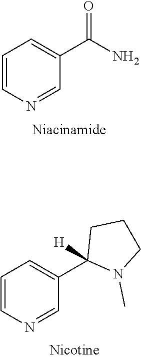

### Vaporization of Niacinamide, Nicotinic Acid, Nicotine, and Cannabinoids.

Niacinamide (nicotinamide) undergoes vaporization from a solid to a vapor at a relatively low 150-160° C., whereas nicotinic acid undergoes sublimation from a solid to a vapor at 236° C. In comparison, nicotine vaporizes at 247° C. and cannabinoids at a much lower, 157-220° C. The vaporization temperature of cannabidiol is between 160° C.-180° C.

Other convenient and effective delivery forms include liquid spray, liquid beverage, liquid “shot”, tincture, gummy or “chewing gum”, chewable, “food-form,” and partially orally-absorbable (e.g., gingival, buccal) soft chew or gummy preparations utilizing mucoadhesive technologies. Topical creams, lotions, salves, balms, “patches”, and other external methods of application could also be employed to deliver the neuroprotective, antioxidant, anti-inflammatory preparation consisting of a broad- or full spectrum of cannabinoids, niacinamide, and/or a blend of synergistic nutrients (e.g., vitamins, minerals) to the skin for dermatological issues related to chronic inflammation such as acne, eczema, dermatitis, psoriasis and other inflammatory skin conditions.

The neurosupportive blend, in some embodiments, is a commercially available blend of organic or conventionally grown hemp oil extract with vitamin B3 (niacinamide) and/or nicotinamide riboside. In some embodiments, the neurosupportive blend comprises: hemp oil extract, 0.5-500 mg; niacinamide, 0.5-250 mg. In some embodiments, the neurosupportive blend comprises: hemp oil extract, 5-500 mg; niacinamide, 5-250 mg. In other embodiments, the neurosupportive blend comprises: hemp oil extract, 0.5-500 mg; nicotinamide riboside, 0.5-500 mg.

The delivery system in some embodiments is a highly specialized combination of the most bioavailable nutrients along with non-GMO tableting agents and excipients which facilitate oral bioavailability through mucosal absorption from within the oral cavity. Some non-limiting examples of such excipient compounds include tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, silicon dioxide, cellulose, stevia (leaf) extract, monk fruit extract, natural or artificial flavors, saccharin, acesulfame, aspartame, neotame, sucralose.

Other such compounds as are known in the art of oral drug absorption and delivery systems may be used. In these and some other embodiments, the neuroprotective preparation is taken as a tablet, soft chew, gummy, chewable wafer, quick-dissolve tablet, or lozenge placed in the mouth. In some embodiments, the lozenge is held under the tongue or placed in a buccal recess and allowed to dissolve. In some embodiments, the soft chew is chewed, allowing exposure of the phytochemicals (e.g., terpenes, phenolics, cannabinoids), bioavailable magnesium, and other nutrients to the microvascular tissue (e.g., sublingual, lingual, and buccal surfaces) of the oral cavity for efficient absorption of the nutrients, and swallowed.

A neuroprotective, antioxidant, anti-inflammatory preparation and method of use has been described. Based upon known mechanisms for unabated oxidative stress (e.g., nutrient deficiencies, suboptimal dietary antioxidant consumption), chronic inflammation, and neuronal cell death secondary to excitotoxicity—the neuroprotective preparation acts to decrease oxidative stress through the innate antioxidant activity of a full-spectrum of terpenes, phenolics, and cannabinoids (from hemp oil), the upregulation of endogenous antioxidant enzyme systems (e.g., superoxide dismutase, SOD; glutathione peroxidase, GPx; catalase), and to militate against development of increased post-synaptic neuronal intracellular calcium levels by acting upon (via modulation and inhibition) NMDA receptors in the post-synaptic cell membrane. The synergistic effects of a broad-spectrum cannabinoid (hemp) extract (i.e. hemp oil) and niacinamide are effectively coupled to create an optimal therapeutic effect while reducing undesirable, adverse effects (i.e. side-effects).

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

Nutritional deficiencies are extremely prevalent today due to a variety of factors. Nutrient-depleted soils, unbalanced diets, increased requirements due to stress, injury, excess refined sugar, caffeine, alcohol consumption, individual idiosyncrasies, poor appetite, dieting to promote weight loss, and pharmaceutical medications can inhibit nutrient absorption and/or increase excretion leading to nutrient deficiencies.

Vitamins and minerals serve as important cofactors and catalysts for thousands of enzymatic reactions and chemical processes in the human body. They are indispensable to life, and in health maintenance and disease prevention, but are often deficient due to a variety of factors.

As briefly discussed, there are many circumstances in which the level of one or more vitamins or minerals may be insufficient leading to compromised biological processes and signs of deficiency. Symptoms indicative of suboptimal nutrient intake (e.g., copper and/or zine) include fatigue, poor stamina, cognitive impairment, cardiovascular dysfunction, skin problems, weakened immune system, and susceptibility to infections. Other common signs of vitamin and/or mineral deficiency (e.g., magnesium, B-vitamins) include digestive disturbances, hair loss, impaired wound healing, and sleep disturbances. Malabsorption of vitamins and minerals is also seen in a variety of conditions. Examples are irritable bowel syndrome, prolonged diarrhea, pernicious anemia, disorders of the liver and digestive system, prolonged diarrhea, and hyperthyroidism. In addition, since a number of vitamins (e.g., biotin, vitamin K2) are provided by the gastrointestinal microbiome, use of antibiotics that destroy beneficial bacterial flora may inevitably lead to decreased intestinal vitamin production.

The wide-ranging effects of nutrient deficiencies on multiple health outcomes has been extensively studied and described in hundreds of international studies. It has been well-documented that several key, neuroprotective, cardioprotective, immune strengthening nutrients are commonly deficient due to mineral-depleted soils and the Standard American Diet (SAD), and are further depleted by chronic stress, physical work, exercise, pregnancy, excess refined sugar, alcohol, caffeine, nicotine, glyphosate (e.g., divalent (2+) ions: magnesium, zinc, iron, copper, manganese, selenium), and various medications that inhibit vitamin absorption (e.g., proton pump inhibitors, antacids, metformin; vitamin B12), and promote the excretion of protective elements (e.g., spironolactone, copper).

Vitamin B3 (e.g., niacinamide) functions as a potent antioxidant in biological systems. As a potent antioxidant, on par or greater than ascorbate and alpha-tocopherol, niacinamide also possesses powerful anti-inflammatory properties. Niacinamide showed significant inhibition of oxidative damage induced by reactive oxygen species (ROS) generated by ascorbate-Fe²⁺ and photosensitization systems in brain mitochondria, and at millimolar concentrations, niacinamide protected against both protein oxidation and lipid peroxidation. However, its effect on the inhibition of protein oxidation was greater than that for lipids. The protective effect was observed at biologically relevant concentrations, with an effect greater than that of the endogenous antioxidants, ascorbic acid and alpha-tocopherol. The results from a 1999 study by Kamat and Devasagayam demonstrate that niacinamide can be viewed as a potent antioxidant capable of protecting cell membranes in the brain against oxidative stress causes by free radical assault.

In addition to its neuroprotective effects on the brain and nervous system, as a powerful antioxidant, topical application of niacinamide has been shown to exhibit anti-inflammatory properties, and improve the tone and texture of the skin, as well as reduce fine lines, wrinkles, and hyperpigmentation.

This invention relates to cognitive-enhancing, stress-reducing, pain-relieving, antioxidant, anti-inflammatory preparations containing niacinamide and broad or full spectrum hemp oil extract.

This invention relates to hemp oil extracts containing a broad or full spectrum of cannabinoids in combination with niacinamide—as the 2-component core—potentially enhanced with selected, synergistic nutrients in a next-generation, antioxidant, anti-inflammatory neuroprotective preparation.

Niacin deficiency. A suboptimal intake of niacin (vitamin B3) can result in the following symptoms of deficiency: fatigue, apathy, depression, headache, and disorientation. Further symptoms of deficiency include dermatitis, swollen mouth and bright red tongue, alopecia, muscle weakness, twitching, burning in the extremities, altered gait, diarrhea, depression, anxiety, progressing to vertigo, memory loss, paranoia, psychotic symptoms, and aggression.

Niacin (vitamin B3) is a water-soluble vitamin whose derivatives such as NAD, NADH, NAD+, and NADP play essential roles in energy metabolism in the living cell and DNA repair. The designation vitamin B3 includes the acid (carboxy) form, nicotinic acid, and the basic (amide) form, nicotinamide or niacinamide. A severe lack of niacin causes the deficiency disease pellagra characterized by dermatitis and dementia, whereas a mild deficiency can present with a wide variety of symptoms such as fatigue, depression, headache, and muscle weakness. The recommended daily allowance of niacin is 2-12 mg a day for children, 14 mg a day for women, 16 mg a day for men, and 18 mg a day for pregnant or breast-feeding women. The liver can synthesize niacin from the essential amino acid tryptophan, but the synthesis is extremely slow and requires vitamin B6; 60 mg of tryptophan are required to make one milligram of niacin. Bacteria in the gut may also perform the conversion but are inefficient. Although nicotinic acid and nicotinamide (niacinamide) are identical in their vitamin activity, nicotinamide does not have the same lipid-modifying effects as nicotinic acid. When niacin takes on the—amide group, it does not reduce cholesterol or cause flushing.

The central redox coenzyme in cellular metabolism, NAD+ functions as a hydride (H⁻) acceptor, forming NADH with simultaneous oxidation of carbohydrate, fat, and protein metabolites. Thus, proper carbohydrate, fat, and protein metabolism requires NAD+ as a hydride acceptor, whereas oxidative phosphorylation, gluconcogenesis, ketogenesis, lipogenesis, and detoxification of reactive oxygen species (ROS) require the reduced co-factors, NADH and NADPH, as hydride donors.

Cannabinoids (from hemp extract) and magnesium exert a myriad of antioxidant, anti-inflammatory benefits on the brain and nervous system, cardiovascular system, skin, bones, and possess wide-ranging, general health benefits.

A neuroprotective, antioxidant, anti-inflammatory preparation and method of use has been described. Based upon known mechanisms for unabated oxidative stress (e.g., nutrient deficiencies, suboptimal dietary antioxidant consumption), chronic inflammation, and neuronal cell death secondary to excitotoxicity—the neuroprotective preparation acts to decrease oxidative stress through the innate antioxidant activity of a full-spectrum of cannabinoids (from hemp extract), upregulation of endogenous antioxidant enzyme systems (e.g., superoxide dismutase, SOD; glutathione peroxidase, GPx; catalase), and to militate against development of increased post-synaptic neuronal intracellular calcium levels by acting upon NMDA receptors in the post-synaptic cell membrane. The synergistic effects of a broad-spectrum cannabinoid (hemp) extract and niacinamide are effectively coupled to create an optimal therapeutic effect while reducing undesirable, adverse effects (i.e. side-effects).

In this disclosure, “about” means within +/−10% of a stated value.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

### REFERENCES

McCance and Widdowson. The Composition of Foods. MAFF and the Royal Society of Chemistry, 1991.
Bergner P. The healing power of minerals, special nutrients and trace elements, p. 312. 1997; Prima Publishing.
Thomas D. The mineral depletion of foods available to us as a nation (1940-2002)—a review of the 6th Edition of McCance and Widdowson. Nutr Health 2007; 19(1-2):21-55.
Samsel A, Seneff S. Glyphosate, pathways to modern diseases II: Celiac sprue and gluten intolerance. Interdiscip Toxicol 2013; 6(4):159-84.
Samsel A, Seneff S. Glyphosate, pathways to modern diseases III: Manganese, neurological diseases, and associated pathologies. Surg Neurol Int 2015; 6:45.
Gillam, Carey. Heavy use of herbicide Roundup linked to health dangers—U.S. study. reuters.com; Apr. 25, 2013.
Choi A L, Zhang Y, Sun G, et al. Association of lifetime exposure to fluoride and cognitive functions in Chinese children: a pilot study. Neurotoxicol Teratol 2015; 47:96-101.
Agalakova N I, Gusev G P, “Molecular Mechanisms of Cytotoxicity and Apoptosis Induced by Inorganic Fluoride,” ISRN Cell Biology, vol. 2012, Article ID 403835, 16 pages.
Sebastian S T, Sunitha S. A cross-sectional study to assess the intelligence quotient (IQ) of school going children aged 10-12 years in villages of Mysore district, India with different fluoride levels. J Indian Soc Pedod Prev Dent 2015; 33(4):307-11.
Nunes M A, Viel T A, Buck H S. Microdose lithium treatment stabilized cognitive impairment in patients with Alzheimer's disease. Curr Alzheimer Res. 2013 January; 10(1):104-107.
Kalghatgi S, Spina C S, Costello J C, et al. Bactericidal antibiotics induce mitochondrial dysfunction and oxidative damage in Mammalian cells. Sci Transl Med. 2013; 5(192): 192ra85.
Singh R, et al. Side effects of antibiotics during bacterial infection: mitochondria, the main target in host cell. Mitochondrion. 2014 May; 16:50-4.
Tobore T O. On the central role of mitochondria dysfunction and oxidative stress in Alzheimer's disease. Neurol Sci. 2019 August; 40(8):1527-1540. doi: 10.1007/s10072-019-03863-x. Epub 2019 Apr. 13.
Schnell D M, et al. Vitamin D produces a perilipin 2-dependent increase in mitochondrial function in C2C12 myotubes. J Nutr Biochem. 2019 March; 65:83-92.
Singla M, et al. Vitamin D supplementation improves simvastatin-mediated decline in exercise performance: A randomized double-blind placebo-controlled study. J Diabetes. 2017 December; 9(12):1100-1106.
Lee S R. Critical Role of Zinc as Either an Antioxidant or a Prooxidant in Cellular Systems. Oxid Med Cell Longev. 2018 Mar. 20; 2018:9156285.
Long A N, Atwell C L, Yoo W, Solomon S S. Vitamin B(12) deficiency associated with concomitant metformin and proton pump inhibitor use. Diabetes Care. 2012 December; 35(12):e84.
Sears M E. Chelation: harnessing and enhancing heavy metal detoxification—a review. Scientific World Journal. 2013; 2013:219840. Published 2013 Apr. 18.
Greenfield, Heather; Southgate, D. A. T. (2003). Food composition data: production, management and use (2 cd.). Rome: Food and Agriculture Organization of the United Nations. p. 146.
Ma J F. Role of organic acids in detoxification of aluminum in higher plants. Plant Cell Physiol. 2000 April; 41(4):383-90.
Ravichandran, S. Possible Natural Ways to Eliminate Toxic Heavy Metals. International Journal of ChemTech Research CODEN(USA): IJCRGG ISSN: 0974-4290 Vol. 3, No. 4, pp 1886-1890 October-December 2011.
Flora S J, Kannan G M, Pant B P, Jaiswal D K. Combined administration of oxalic acid, succimer, and its analogue for the reversal of gallium arsenide-induced oxidative stress in rats. Archives of Toxicology. 2002; 76(5-6):269-276.
Gasperi V, Sibilano M, Savini I, Catani M V. Niacin in the Central Nervous System: An Update of Biological Aspects and Clinical Applications. Int J Mol Sci. 2019; 20(4):974.
Jennifer Margham, et al. Chemical Composition of Aerosol from an E-Cigarette: A Quantitative Comparison with Cigarette Smoke. Chemical Research in Toxicology. 2016 29 (10), 1662-1678.
Meng Y, Ren Z, Xu F, et al. Nicotinamide Promotes Cell Survival and Differentiation as Kinase Inhibitor in Human Pluripotent Stem Cells. Stem Cell Reports. 2018; 11(6):1347-1356.
Son M J, Son M Y, Seol B, Kim M J, Yoo C H, Han M K, Cho Y S. Nicotinamide overcomes pluripotency deficits and reprogramming barriers. Stem Cells. 2013 June; 31(6):1121-35.
Levenson C W, Morris D. Zinc and neurogenesis: making new neurons from development to adulthood. Adv Nutr. 2011; 2(2):96-100.
Kamat J P, Devasagayam T P. Nicotinamide (vitamin B3) as an effective antioxidant against oxidative damage in rat brain mitochondria. Redox Rep. 1999; 4(4): 179-84.
Bissett D L, Oblong J E, Berge C A. Niacinamide: a B vitamin that improves aging facial skin appearance. Dermatol Surg 31(7 Pt 2):860-5 (2005 July).

Although the invention has been explained several embodiments, it is to be understood that many other possible modifications and variations can be made without departing from the spirit and scope of the invention.

What is claimed is:

1. A composition comprising 100-5,000 milligrams glycine, 100-2,000 milligrams taurine, 100-2,000 milligrams malic acid, and 10-400 milligrams magnesium.

2. The composition of claim 1, wherein the composition comprises a broad-spectrum hemp oil extract, magnesium, and a base of synergistic (e.g., antioxidant, neuroprotective, cardioprotective, immunosupportive) vitamins, minerals, and other nutrients or nutraceuticals.

3. The composition of claim 1, wherein the composition comprises a full spectrum of naturally occurring cannabinoids and phytochemicals obtained from hemp oil extract, magnesium, and a base of synergistic (e.g., antioxidant, neuroprotective, cardioprotective, immunosupportive) vitamins, minerals, and other nutrients or nutraceuticals.

4. The composition of claim 1, wherein said magnesium is either inorganic or organic magnesium.

5. The composition of claim 4, wherein the magnesium compound or chelate comprises a counterion or chelating compound selected from the group consisting of oxide, sulfate, carbonate, chloride, succinic acid, ascorbic acid, aspartic acid, citric acid, gluconic acid, threonic acid, lysinic acid, glycine, malic acid, or taurine.

6. The composition of claim 5, wherein the magnesium chelate comprises a chelating compound selected from the group of glycine, malic acid, or taurine.

7. The composition of claim 4, wherein the magnesium is elemental magnesium.

8. The composition of claim 5, wherein the magnesium is provided as malate, glycinate, taurinate, or threonate for maximum therapeutic efficacy.

9. The composition of claim 1, with the base of synergistic vitamins, minerals, and other nutrients including more specifically (per serving): vitamin A as retinol, retinyl palmitate, retinyl acetate, or beta-carotene, 250-10,000 iu; vitamin B1, 0.25-100 mg; vitamin B2, 0.25-100 mg; vitamin B3, 5-250 mg; vitamin B5, 5-1,000 mg; vitamin B6, 0.25-100 mg; vitamin B12, 5-5,000 mcg; biotin, 5-1,000 mcg; PABA, 0.50-100 mg; vitamin C, 50-5,000 mg; vitamin D2 or D3, 400-10,000 IU; natural vitamin E (tocopherols and/or tocotrienols), 5-1,000 iu; vitamin K2 (Mk-7), 5-500 mcg; boron (glycinate), 0.1-10 mg; copper (glycinate), 0.1-5 mg; zinc (glycinate), 2-50 mg; manganese (glycinate), 0.1-10 mg; selenium (glycinate), 5-400 mcg; molybdenum (glycinate), 5-400 mcg; chromium (nicotinate glycinate or polynicotinate), 5-1,000 mcg; and kelp (as a source of natural iodine), 5-200 mg.

10. The composition of claim 9, with the addition of one or more of the following synergistic nutraceuticals or phytochemicals (per serving): coenzyme Q10 as ubiquinone or ubiquinol, 2.0-1,000 mg; alpha lipoic acid, 2.0-1,000 mg; Extramel French melon extract, 0.5-20 mg, AuroraBlue Alaskan Blueberry Concentrate or Extract, 5-5,000 mg; hemp oil extract, 1-1,000 mg supplying 0.5-100 mg of cannabidiol (CBD) and other cannabinoids, terpenes, and phenolics.

11. The composition of claim 10, comprising iodine as potassium iodide, 50-500 mcg; vitamin A as retinol or retinyl palmitate or acetate or beta-carotene, 500-5,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 3-5,000 mcg as cyanocobalamin or methylcobalamin or hydroxocobalamin or adenosylcobalamin; folic acid or methyl-folate or folinic acid, 10-1,000 mcg; 25-1,000 mcg; vitamin C, 25-1,000 mg; vitamin D2 or D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; vitamin K2 (Mk-7), 5-500 mcg; boron as glycinate (as well as other organic or inorganic forms of the mineral), 0.1-10 mg; copper as bisglycinate (as well as other organic or inorganic forms of the mineral), 0.1-5 mg; zinc as bisglycinate (as well as other organic or inorganic forms of the mineral), 2-50 mg; manganese as bisglycinate (as well as other organic or inorganic forms of the mineral), 0.1-10 mg; selenium as glycinate (as well as other organic or inorganic forms of the mineral), 5-200 mcg; molybdenum as glycinate (as well as other organic or inorganic forms of the mineral), 5-400 mcg; and chromium as nicotinate glycinate or polynicotinate (as well as other organic or inorganic forms of the mineral), 5-1,000 mcg.

12. The composition of claim 1, comprising vitamin A as retinol or retinyl palmitate or acetate or beta-carotene, 500-5,000 iu; vitamin B1, 0.5-25 mg; vitamin B2, 0.5-25 mg; vitamin B3, 0.5-50 mg; vitamin B5, 10-100 mg; vitamin B6, 0.5-25 mg; vitamin B12, 10-1,000 mcg as methylcobalamin or hydroxocobalamin or adenosylcobalamin; folic acid or methyl-folate or folinic acid, 100-400 mcg; vitamin C, 25-1,000 mg; vitamin D2 or D3, 500-2,000 IU; vitamin E as d-alpha tocopheryl succinate and/or mixed tocopherols and/or tocotrienols, 30-100 iu for alpha-tocopherol and 5-500 mg for mixed tocopherols including gamma-tocopherol or the family of tocotrienols; vitamin K2 (Mk-7), 45-200 mcg; boron as glycinate (as well as other organic or inorganic forms of the mineral), 0.25-5 mg; copper as bisglycinate (as well as other organic or inorganic forms of the mineral), 0.25-2 mg; zinc as bisglycinate (as well as other organic or inorganic forms of the mineral), 5-20 mg; manganese as bisglycinate (as well as other organic or inorganic forms of the mineral), 0.5-5 mg; selenium as glycinate (as well as other organic or inorganic forms of the mineral), 25-200 mcg; molybdenum as glycinate (as well as other organic or inorganic forms of the mineral), 25-200 mcg; and chromium as nicotinate glycinate (as well as other organic or inorganic forms of the mineral), 25-200 mcg; and kelp (as a source of natural iodine), 5-200 mg.

13. The composition of claim 1, further comprising a soft chew, gummy, or tableting agent and an excipient that facilitates oral bioavailability through mucosal absorption from within the oral cavity.

14. The composition of claim 13, wherein said excipient that facilitates oral bioavailability through mucosal absorption from within the oral cavity comprises one or more of tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, silicon dioxide, cellulose, stevia (leaf) extract, monk fruit extract, natural or artificial flavors, saccharin, acesulfame, aspartame, neotame, sucralose.

15. A composition comprising hemp oil extract containing of about between 0.5 and 500 mg, and a cannabidiol content of about between 0.25 and 100.0 milligrams, and niacinamide in an amount of about between 1.0 and 250 milligrams per serving.

16. The composition of claim 15, comprising hemp oil extract of about between 12.5 and 500 mg, and a cannabidiol content of about between 1.25 and 50 milligrams, and niacinamide in an amount of about between 5 and 100 milligrams per serving.

17. The composition of claim 15, comprising hemp oil extract of about between 25 and 250 mg, and a cannabidiol content of about between 2.5 and 50 milligrams, and niacinamide in an amount of about between 10 and 100 milligrams per serving.

18. The composition of claim 15, comprising hemp oil extract of about between 0.5 and 500 milligrams, and niacinamide of about between 2 and 250 milligrams, and/or nicotinamide riboside of about between 0.5 and 500 mg per serving.
